(19) United States
(12) Patent Application Publication
(10) Pub. No.: US 2025/0295696 A1
(43) Pub. Date: Sep. 25, 2025

(54) T-CELL TARGET DISCOVERY

(71) Applicant: 3T BIOSCIENCES, INC., South San Francisco, CA (US)

(72) Inventors: Marvin Gee, San Francisco, CA (US); Leah Sibener, San Francisco, CA (US)

(21) Appl. No.: 18/862,877

(22) PCT Filed: May 3, 2023
(86) PCT No.: PCT/US2023/020799

§ 371 Date: Nov. 4, 2024

Related U.S. Application Data
(60) Provisional application No. 63/337,975, filed on May 3, 2022.

(51) Int. Cl. A61K 35/17; A61K 40/11; A61K 40/32; A61K 45/06; C12N 15/10; G01N 33/50; G16B 15/30; G16H 20/17

(52) U.S. Cl. CPC A61K 35/17; A61K 40/11; A61K 40/32; A61K 45/06; C12N 15/1037; G01N 33/5011; G01N 33/505; G16B 15/30; G16H 20/17; G01N 2333/7051

(57) ABSTRACT

The present invention provides methods and systems that identify novel antigens that bind to a particular T cell receptor and also validate the immunogenicity of the potential antigens to activate the TCR. The methods allow for development of an exhaustively profile of on-target and off-target reactivity of novel antigens.

### FIELD OF DISCLOSURE

This disclosure relates methods for identifying targets for optimized antigen reactive T-cells.

### BACKGROUND

Cancers are attributed to nearly 10 million deaths globally each year. Although recent advances in drug therapies have improved patient outcomes in some cancers, due to the complexity and heterogeneity of cancer cells there is no guarantee that any particular drug therapy will successfully result in remission and control of a patient's cancer. Moreover, remission and control can be fleeting, with drug targets changing as cancer cells continue to mutate and develop resistances to previously effective therapies.

Diseases and disorders of the immune system are another significant cause of human illness. There are numerous reasons the immune system may fail to function. For example, the immune system may underreact or overreact to foreign antigens. In autoimmune diseases, the immune system may target normal or heathy tissues, for example by targeting the epitopes presented by cells of the human body.

Engineered immune cells have been proposed as potential treatment for cancers and other antigen presenting maladies. However, these immune cells, whether chimeric antigen receptor (CAR)-engineered cells or T-cell receptor (TCR)-engineered cells, require specificity for disease associated antigens. Unfortunately, increasing the affinity of TCRs in engineered T-cells to known disease antigens frequently increases the affinity of the cells to non-disease-specific peptides, resulting in severe and intolerable side effects. Moreover, many epitopes of disease antigens may be patient specific or specific to a narrow population, preventing identification of optimal disease targets.

As a consequence, dangerous cross-reactivity of engineered T-cells has halted development of therapeutics products even where cross-reactivity for the TCR was not predicted. Thus, despite decades of consistent research, engineered T-cell specific therapies have struggled to find regulatory approval.

### SUMMARY

Provided are methods for identifying novel epitopes that are targetable by immune cell therapies. Methods of the invention assay immune cells from individuals afflicted with an immune mediated condition, for example cancers or auto-immune disorders. The T-cell receptors (TCRs) of T-cells from the subject may then be identified and analyzed. Once analyzed, the TCRs may be engineered and assayed against peptide libraries of predicted epitopes of the TCRs. The predicted epitopes may then be validated as epitopes of the T-cell, and thereby validated as epitopes indicative of the subject's disease condition. Advantageously, the methods of the invention utilize the immune system of subject afflicted with a disease condition to identify epitopes indicative of the disease that are then targetable by engineered T-cell therapies.

Aspects of the invention provide a method that comprises identifying T-cell receptors of immune cells from sequencing data obtained from a subject. The sequencing data is then used to engineer a soluble TCR or T-cell expressing a TCR identified from the sequencing data. The engineered TCRs are then screened against a peptide library expressing predicted epitopes of the TCR. The predicted epitopes in the library are then validated as epitopes of the T-cell and thereby epitopes of the disease condition. By the methods of the invention, novel epitopes, for example patient specific and patient group specific epitopes, are identified with improved affinity and/or reduced cross-reactivity to known epitopes.

The immune cells may be any immune cells obtained from a subject with an antigen presenting disorder. For example, the immune cells may be from a subject afflicted with cancer or an auto-immune disorder. Where the disorder is cancer, the immune cells from the subject may be from tumor- or tissue-infiltrating lymphocytes. The immune cells may be from peripheral blood mononuclear cells (PBMCs). The immune cells may be obtained from the tumor of a subject previously administered a cancer therapy. The immune cells may be obtained from a subject that is a responder or a non-responder to the cancer therapy. The cancer therapy may comprise one or more of an immune checkpoint inhibitor, neoadjuvant therapy, and/or chemotherapy.

Where the epitopes are epitopes of the cancer, the immune cells may be obtained from a tumor associated with one or more cancers selected from the group comprising breast cancer, cervical cancer, colorectal cancer, endometrial cancer, glioma, head and neck cancer, liver cancer, lung cancer, lymphoma, melanoma, ovarian cancer, pancreatic cancer, ovarian cancer, pancreatic cancer, prostate cancer, renal cancer, skin cancer, stomach cancer, testis cancer, thyroid cancer, and urothelial cancer.

In aspects of the invention a differential analysis may be performed. For example, the immune cells may be obtained from a subject prior to administration of the cancer therapy and after administration of the cancer therapy. The methods of the invention may then be performed separately on the two immune cells samples and a differential analysis of the epitopes of TCRs pre-therapy and post-therapy may be performed.

The methods of invention use sequencing data to engineer a soluble TCR or T-cell expressing a TCR identified from sequencing data of immune cells. The engineered TCRs are then screened against a peptide library expressing predicted epitopes of the TCR. The peptide library may be any known peptide library, for example a yeast display library. Peptide libraries that may be used with the present invention include those described in PCT Publication Nos. WO 2015/153969, WO 2020/047502, and WO 2021/168388; U.S. Publication No. 2021-0309993; and U.S. Pat. Nos. 10,816,554, 11,125,755, and 11,125,756, the entirety of the contents of each of which are incorporated by reference herein.

The peptide libraries benefit from displaying predicted epitopes of the TCR. The predicted epitopes may be predicted by a machine-learning algorithm or statistical algorithm as described further hereinbelow. Advantageously, the peptide library may comprise predicted epitopes selected from one or more of wildtype human sequences, patient-specific neoantigens, shared neoantigens, spliced peptides, human endogenous retroviruses (hER Vs), long interspersed nuclear elements (LINEs), aeTSAs (aberrantly expressed, tumor specific antigens), frameshifts, gene fusions, alternative splicing, aberrant translations, alternative promoters, human-viral targets, and human-bacterial targets.

The peptide library may express peptides of any length. In aspects of the invention the peptide library may comprise peptides that are 8-11 mer peptides, for example 8-mer, 9-mer, 10-mer, or 11-mer peptides.

Advantageously, immune cells may be obtained, prepared, and analyzed by any known methods. For example, the immune cells may be obtained from formalin fixed paraffin-embedded tissue.

Methods of the invention comprise validating predicted epitopes as epitopes of the obtained T-cells. The validating step may comprise analyzing epitope/T-cell affinity by any known methods. For example, the validating step may comprise analyzing T-cell activation (for example, CD69 activation), T-cell killing, mass spectrometry, functional antigen procession, and/or target expression. For example, the validating step comprises analyzing T-cell killing of cells expressing the peptide by an engineered T-cell comprising the TCR.

The present invention provides methods and systems that identify novel antigens that bind to a particular T cell receptor and also validate the immunogenicity of the potential antigens to activate the TCR. The methods allow for development of an exhaustively profile of on-target and off-target reactivity of novel antigens. The systems and methods are also able to predict efficacy and safety profile of immune cells activated by particular antigens.

TCRs and their cognate peptide-HLA targets, e.g., peptide-Major Histocompatibility Complexes (pMHC), possess inherent variability across receptors and antigens. The variability among the components of various TCR-pMHC systems means that determining the antigen specificity for a TCR has poses a complex problem. The presently disclosed systems and methods provide a high-throughput path through this bottleneck. By combining computational and wet lab techniques, the systems and methods of the invention provide an unprecedentedly accurate and exhaustive profile of epitopes targeted by the TCRs of immune cells from subjects with an antigen presenting disorder. The activity of particular T cell receptors to determine their specificities and/or cross-reactivities to antigens allows for the identification of novel target antigens in patient populations with reduced cross-reactivities to other compounds or receptors when targeted. As explained in greater detail herein, the systems and methods of the invention may provide direct identification of novel antigens that bind to a TCR, without any a priori knowledge of the antigens.

Accordingly, aspects of the present invention provide methods for treating a subject having an antigen-presenting disorder.

The methods include treating a subject afflicted with cancer or an auto-immune disorder by providing to the subject a composition comprising an engineered T-cell or soluble TCR targeting a first epitope, wherein the first epitope was identified by the steps of identifying T-cell receptors (TCRs) of immune cells from sequencing data obtained from a subject, engineering a soluble TCR or T-cell expressing a TCR identified from the sequencing data, screening the soluble TCR or engineered T-cell against a peptide library expressing predicted epitopes of the TCR of the engineered T-cell including the first epitope, and validating the first epitope from the peptide library as an epitope of the T-cell.

The immune cells may be any immune cells obtained from a subject with an antigen presenting disorder. For example, the immune cells may be from a subject afflicted with cancer or an auto-immune disorder. Where the disorder is cancer, the immune cells from the subject may be from tumor- or tissue-infiltrating lymphocytes. The immune cells may be from peripheral blood mononuclear cells (PBMCs). The immune cells may be obtained from the tumor of a subject previously administered a cancer therapy. The immune cells may be obtained from a subject that is a responder or a non-responder to the cancer therapy. The cancer therapy may comprise one or more of an immune checkpoint inhibitor, neoadjuvant therapy, and/or chemotherapy.

Where the epitopes are epitopes of the cancer, the immune cells may be obtained from a tumor associated with one or more cancers selected from the group comprising breast cancer, cervical cancer, colorectal cancer, endometrial cancer, glioma, head and neck cancer, liver cancer, lung cancer, lymphoma, melanoma, ovarian cancer, pancreatic cancer, ovarian cancer, pancreatic cancer, prostate cancer, renal cancer, skin cancer, stomach cancer, testis cancer, thyroid cancer, and urothelial cancer.

In aspects of the invention a differential analysis may be performed. For example, the immune cells may be obtained from a subject prior to administration of the cancer therapy and after administration of the cancer therapy. The methods of the invention may then be performed separately on the two immune cells samples and a differential analysis of the epitopes of TCRs pre-therapy and post-therapy may be performed.

The methods of invention use sequencing data to engineer a soluble TCR or T-cell expressing a TCR identified from sequencing data of immune cells. The engineered TCRs are then screened against a peptide library expressing predicted epitopes of the TCR. The peptide library may be any known peptide library, for example a yeast display library.

The peptide libraries benefit from displaying predicted epitopes of the TCR. The predicted epitopes may be predicted by a machine-learning algorithm or statistical algorithm as described further hereinbelow. Advantageously, the peptide library may comprise predicted epitopes selected from one or more of wildtype human sequences, patient-specific neoantigens, shared neoantigens, spliced peptides, human endogenous retroviruses (hERVs), long interspersed nuclear elements (LINEs), aeTSAs (aberrantly expressed, tumor specific antigens), frameshifts, gene fusions, alternative splicing, aberrant translations, alternative promoters, human-viral targets, and human-bacterial targets.

The peptide library may express peptides of any length. In aspects of the invention the peptide library may comprise peptides that are 8-11 mer peptides, for example 8-mer, 9-mer, 10-mer, or 11-mer peptides.

Advantageously, immune cells may be obtained, prepared, and analyzed by any known methods. For example, the immune cells may be obtained from formalin fixed paraffin-embedded tissue.

Methods of the invention comprise validating predicted epitopes as epitopes of the obtained T-cells. The validating step may comprise analyzing epitope/T-cell affinity by any known methods. For example, the validating step may comprise analyzing T-cell activation (for example, CD69 activation), T-cell killing, mass spectrometry, functional antigen procession, and/or target expression. For example, the validating step comprises analyzing T-cell killing of cells expressing the peptide by an engineered T-cell comprising the TCR.

### BRIEF DESCRIPTION OF THE DRAWINGS

FIG. 1 depicts a method of creating a library of predicted epitopes.

FIG. 2 depicts a flow chart of methods of the invention.

### DETAILED DESCRIPTION

The present invention provides methods and systems that identify novel antigens that bind to a particular T cell receptor and also validate the immunogenicity of the potential antigens to activate the TCR. The methods allow for development of an exhaustively profile of on-target and off-target reactivity of novel antigens.

### Definitions

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The term “major histocompatibility complex” (MHC) proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II. MHC proteins as used herein includes MHC proteins from any mammalian or avian species, e.g. primate sp., particularly humans; rodents, including mice, rats and hamsters; rabbits; equines, bovines, canines, felines, etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. Included in the murine H-2 subunits are the class I H-2K, H-2D, H-2L, and the class II I-Aα, I-Aβ, I-Eα and I-Eβ, and β2-microglobulin.

As used herein, the term “class II HLA/MHC” binding domains comprise the α1 and α2 domains for the a chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind target peptides (i.e., peptide ligands). Class II HLA/MHC binding domains also refers to the binding domains of a major histocompatibility complex protein that are soluble domains of Class II α and β chain. Class II HLA/MHC binding domains include domains that have been subjected to mutagenesis and selected for amino acid changes that enhance the solubility of the single chain polypeptide, without altering the peptide binding contacts.

As used herein, the term “class I HLA/MHC” binding domains includes the α1, α2 and α3 domain of a Class I allele, including without limitation HLA-A, HLA-B, HLA-C, H-2K, H-2D, H-2L, which are combined with β2-microglobulin. Not more than about 10, usually not more than about 5, preferably none of the amino acids of the transmembrane domain will be included. The deletion will be such that it does not interfere with the ability of the domains to bind target peptides (i.e., peptide ligands).

The “MHC binding domains”, as used herein, refers to a soluble form of the normally membrane-bound protein. The soluble form is derived from the native form by deletion of the transmembrane domain. The MHC binding domain protein is truncated, removing both the cytoplasmic and transmembrane domains and includes soluble domains of Class II alpha and beta chain. “MHC binding domains” also refers to binding domains that have been subjected to mutagenesis and selected for amino acid changes that enhance the solubility of the single chain polypeptide, without altering the peptide binding contacts.

“MHC context” as used herein refers to an interaction being in the presence of an MHC with non-covalent interactions with the MHC and an antigen. The function of MHC molecules is to bind peptide fragments derived from pathogens and display them on the cell surface for recognition by the appropriate T cells. Thus, TCR recognition can be influenced by the MHC protein that is presenting the antigen. The term MHC context refers to the recognition by a TCR of a given peptide, when it is presented by a specific MHC protein.

“T cell receptor” (TCR), refers to an antigen/MHC binding heterodimeric protein product of a vertebrate (e.g., mammalian, TCR gene complex, including the human TCR α, β, γ, and δ chains). For example, the complete sequence of the human β TCR locus has been sequenced, as published by Rowen 1996; the human TCR locus has been sequenced and resequenced, for example, see Mackelprang 2006; see a general analysis of the T-cell receptor variable gene segment families in Arden 1995; each of which is herein specifically incorporated by reference for the sequence information provided and referenced in the publication.

TCRs used in the present invention may be bispecific TCRs. For example, the TCR may comprise at one end a soluble TCR with specificity for a target antigen and at the other end a fragment that binds and activates T-cells. For example, the fragment may comprise a CD3-directed single-chain variable fragment.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “epitope” as used herein comprises the terms “structural epitope” and “functional epitope”. The “structural epitope” are those amino acids of the antigen, e.g. peptide-MHC complex, that are covered by the antigen binding protein when bound to the antigen. Typically, all amino acids of the antigen are considered covered that are within 5 A of any atom of an amino acid of the antigen binding protein. The structural epitope of an antigen may be determined by art known methods including X-ray crystallography or NMR analysis. The structural epitope of an antibody typically comprises 20 to 30 amino acids. The structural epitope of a TCR typically comprises 20 to 30 amino acids. The “Functional Epitope” is a subset of those amino acids forming the structural epitope and comprises the amino acids of the antigen that are critical for formation of the interface with the antigen binding protein of the invention, either by directly forming non-covalent interactions such as H-bonds, salt bridges, aromatic stacking or hydrophobic interactions or by indirectly stabilizing the binding conformation of the antigen and is, for instance, determined by mutational scanning. The term “epitope” includes any molecule, structure, amino acid sequence, or protein determinant that is recognized and specifically bound by a cognate binding molecule, such as a chimeric antigen receptor, or other binding molecule, domain, or protein.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are substituted, mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound.

In certain embodiments, a functional variant or functional portion or functional fragment each refers to a “signaling portion” of an effector molecule, effector domain, costimulatory molecule, or costimulatory domain. In other aspects, a functional variant or functional portion or functional fragment each refers to a linking function or a leader peptide function as disclosed herein. In certain aspects, a functional variant/portion/fragment refers to a linking function or a leader peptide function as described herein. In specific aspects, variant linkers and leader peptides are at least 60% as efficient, at least 70% as efficient, at least 80% as efficient, at least 90% as efficient, at least 95% as efficient, or at least 99% as efficient as the reference/parent polypeptides disclosed herein.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

Editing a cell means altering the gene expression of the cell. Any known method for editing the gene expression of a cell may be used in combination with methods of the invention. For example, editing may comprise transfection with a vector, electroporation, recombination (e.g., homologous recombination), transformation, transduction, or gene editing (e.g., introducing a CRISPR-Cas9 system, a TALEN system, or a ZNF system into cells).

An exemplary editing system comprises a nuclease and a guide RNA. For example, a CRISPR system comprises a CRISPR nuclease (e.g., CRISPR (clustered regularly interspaced short palindromic repeats)-associated (Cas) endonuclease or a variant thereof, such as Cas9) and a guide RNA. The CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) or sgRNA (synthetic guide RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” is a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. For example, the vector may be a lentivirus or an adenovirus. Here, “plasmid,” “expression plasmid,” “virus,” and “vector” are often used interchangeably.

The terms “modify,” “modifying,” or “modification” in the context of making alterations to nucleic compositions of a cell, and the term “introduced” in the context of inserting a nucleic acid molecule into a cell, include reference to the alteration or incorporation of a nucleic acid molecule in a eukaryotic cell wherein the nucleic acid molecule may be incorporated into the genome of a cell and converted into an autonomous replicon. “Modification” or “introduction” of nucleic compositions in a cell may be accomplished by a variety of methods known in the art, including, but not limited to, transfection, transformation, transduction, or gene editing. As used herein, the term “engineered,” “recombinant,” “modified,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications and/or introductions of expressible nucleic acid molecules encoding polypeptide, such as additions, deletions, substitutions, mutations, or other functional changes of a cell's genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4+ cells while depleting CD8+ cells, or enriching for CD8+ cells while depleting CD4+ cells, or combinations thereof.

“Antigen” as used herein refers to an immunogenic molecule that provokes an immune response. This immune response may involve antibody production, activation of specific immunologically-competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof.

Antigens can be produced by cells that have been modified or genetically engineered to express an antigen.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species (i.e., a heterologous nucleic acid). Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, for example, non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

Any cell assay systems may be used in combination with the assays of the invention. For example, cells may be first separated into reaction chamber, for example using a droplet separation system. Cells for example a microplate comprising 6, 12, 24, 48, 96, 384 or 1536. Cells may be separated, and each cell subjected to separate culture conditions. For example, in validation, cross-reactivity, and optimization assays, each separated cell may be cultured with a different peptide to analyze peptide responses.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, MA) as described in U.S. application Ser. No. 11/167,046, and U.S. Pat. Nos. 7,501,245; 7491498; 7,276, 720; and in U.S. Patent Application Publication Nos. US20090061439; US20080087826;US20060286566; US20060024711; US20060024678; US20080213770; and US20080103058; 3) Applied Biosystems (e.g. SOLID sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference.

Antigen binding region (ABR). As used herein, the term ABR refers to a combination of variable heavy (VH and variable light (VL) polypeptides to associate to form a variable region domain. An ABR is the minimum antibody fragment that contains a complete antigen-recognition and binding site. This region consists of heavy- and one light-chain variable domain in tight, noncovalent association, as a single polypeptide or as a dimer. It is in this configuration that the three CDRs of each variable domain interact to define an antigen-binding site on the surface of the domain. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody dependent cellular toxicity.

A “T cell” or “T lymphocyte” is an immune system cell that matures in the thymus and produces TCRs. T cells can be naïve (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM).

T2 T cells are a subpopulation of T cells that generally express low amounts of HLA-A2 on the cell surface and are thought to only present exogenous peptides. Binding of exogenous peptides to HLA-A2 stabilizes the HLA-A2-peptide complexes.

CD8-positive T cells are a subpopulation of MHC class I-restricted T cells and are mediators of adaptive immunity. They include cytotoxic T cells, which are important for killing cancerous or virally infected cells, and CD8-positive suppressor T cells, which restrain certain types of immune response.

“CD69” is one of the earliest cell surface antigens expressed by T cells following activation. Once expressed, CD69 acts as a costimulatory molecule for T cell activation and proliferation. In addition to mature T cells, CD69 is inducibly expressed by immature thymocytes, B cells, natural killer (NK) cells, monocytes, neutrophils and eosinophils, and is constitutively expressed by mature thymocytes and platelets.

### Yeast Display Libraries

Yeast-display libraries may be prepared and used in, for example, as provided in PCT international application publications WO 2015/153969, WO 2018/175585, WO 2020/047502, and WO 2021/168388, each of which is incorporated herein by reference.

In certain aspects, a library of single chain polypeptides is generated. Each polypeptide may include the binding domains of a major histocompatibility complex (MHC) protein and diverse peptide ligands. The library is initially generated as a population of polynucleotides encoding the single chain polypeptide operably linked to an expression vector, which library may comprise at least 10⁶, at least 10⁷, or as is most common, at least 10⁸ different peptide ligand coding sequences, and may contain up to about 10¹³, 10¹⁴ or more different ligand sequences. Polypeptides from the library are introduced into suitable host cells, which in turn expresses the encoded polypeptide. Preferably, the host cells are yeast cells. The number of unique host cells expressing the polypeptide is generally less than the total predicted diversity of polynucleotides, e.g., up to about 5×10⁹ different specificities, up to about 10⁹, up to about 5×10⁸, up to about 10⁸, etc.

Preferably, the peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. As a fully random library may represent a large number of possible combinations, in preferred aspects, the peptide ligand diversity is limited at the residues that anchor the peptide to the MHC binding domains, which are referred to herein as MHC anchor residues. The position of the anchor residues in the peptide may be determined by specific MHC binding domains. For example, class I binding domains have anchor residues at the P2 position, and at the last contact residue. Class II binding domains have an anchor residue at P1, and depending on the allele, at one of P4, P6 or P9.

A peptide may be provided as short antigenic sequence active in stimulating T cells; or may be provided in the form of the larger protein, e.g., an intact domain, a soluble protein portion, a complete protein, etc. In certain aspects, peptide antigens are identified that are shared between patients and provide a means for broadly applicable therapy. In other aspects identification of antigens provides for a personalized medicine approach

Each yeast displays a unique ligand peptide that is genetically encoded. A typical library contains ˜10⁸ to 10⁹ unique peptides, which are selected by a TCR of interest. The libraries have theoretical nucleotide diversities dictated by the peptide length and library composition. The functional diversity represents the true capacity of the physical libraries based on yeast colony counting after limiting dilution of the library. The displayed peptides may be fragments of naturally occurring antigenic proteins, may be fragments of neoantigenic proteins that are the subject of somatic mutation during tumorigenesis, and/or may be a synthetically generated mimic of an antigenic protein. The synthetic peptides can act as highly potent agonists of T cell receptors. A peptide may be provided as short antigenic sequence active in stimulating T cells; or may be provided in the form of the larger protein, e.g., an intact domain, a soluble protein portion, a complete protein, etc. In some embodiments, peptide antigens are identified that are shared between patients and provide a means for broadly applicable therapy. In other embodiments identification of antigens provides for a personalized medicine approach.

A pHLA yeast-display library may be prepared, in which yeast cells express different introduced protein ligands. A TCR of interest, such as an orphan TCR from a TIC, is introduced to the yeast-display library cells. In certain aspects, the TCR of interest is multimerized to enhance binding, and used to select for host cells expressing those introduced protein ligands that bind to the TCR of interest. Iterative rounds of selection are performed, i.e., the cells that are selected in the first round provide the starting population for the second round. Usually at least three and more usually at least four rounds of selection are performed.

Yeast may be enriched, for example, using an affinity-based selection using a bead-multimerized TCR and grown for iterative rounds of selection. This causes the ligand peptides with high affinity for the TCR to be successively enriched across the rounds of selection, and all yeast DNA is deep-sequenced.

In certain aspects, these synthetic peptide sequences are used to generate a model to make predictions for TCR ligands derived from the human proteome and/or patient-specific exome.

In certain aspects, the present invention provides a method of determining the set of polypeptide ligands that bind to a T cell receptor of interest, comprising the steps of: performing multiple rounds of selection of a polypeptide library as set forth herein with a T cell receptor of interest; performing deep sequencing of the peptide ligands that are selected; inputting the sequence data to computer readable medium, where it is used to generate a search algorithm embodied as a program of instructions executable by computer and performed by means of software components loaded into the computer.

Thus, the present invention also provides software products tangibly embodied in a machine-readable medium. The software product may include instructions operable to cause one or more data processing apparatus to perform operations comprising: obtaining peptide sequences from a yeast-display library after each round of selection; clustering the peptide sequence reads after each round; producing a cluster specific probability position matrix for each cluster; obtain one or more ligand consensus sequence. The probability position matrix for each cluster may be used to compare the yeast-display peptide ligands with one or more reference proteome databases. For example, the proteome databases may include one or more of peptide sequences for neoantigens, wildtype peptides, spliced peptides, human endogenous retroviruses (hERV), aeTSAs (aberrantly expressed TSAs), frameshift mutations, gene fusions, alternative splicing mutations, aberrant translations, and/or an alternative promoter sequence expressed by a cell from the tumor tissue.

The yeast-display library may be obtained after each round of selection via next generation deep sequencing and provided in a FASTQ format. The analysis pipeline includes pre-processing, which may include, for example, demultiplexing and parsing the sequence reads to identify the peptide sequences of the displayed peptides (mimotopes). The sequences of peptides may be determined in module 1 by any convenient methods of high throughput sequencing. In certain aspects, the peptide ligands include ligands of varying sizes. Thus, in certain methods of the invention, peptides may be parsed based on peptide length.

The resulting Sequences may be analyzed, for example, by using clustering algorithms and/or methods. In preferred aspects, the clustering method/algorithm is minimal common oncology data element (mCODE). In certain aspects, clustering the reads includes calculating the reverse hamming distance between all peptides for which sequences were obtained. Reverse hamming distances are hamming distances subtracted from the total length of a peptide and represent the number of shared amino acids between two peptides. They may be calculated using Matlab (Mathworks Inc.) by iterating through each peptide against all other peptides selected during a round. The output score generated is the number of matching amino acid positions between peptides. Based on the reverse hamming distances, peptides may be clustered using mCODE/Cytoscape.

After each round of selection, the identified yeast-display library peptide sequences may be clustered using mCODE. The mCODE clustering condenses the signal, increases the robustness of potential ligand predictions, and reduces the noise relative to other clustering modalities. mCODE uses vertex weighting and weighs nodes based on local neighborhood density. Nodes with high weights are selected as seed nodes of initial clusters. The clusters are augmented by outwardly traversing from the seeds.

The cluster position probability matrices (c-PPM) may be created using the peptides in each cluster. In certain aspects, generating the c-PPM for each cluster includes, scoring the yeast-display library peptides in each cluster using a PPM. The resulting PPM for cach cluster may be fed to an algorithm (conspred algorithm) that merges the PPM outputs using cumulative cluster weighting. The resulting conspred sequences (predicted consensus sequence(s)) for the yeast-display peptides may be used to search one or more proteome databases (e.g., for human proteins (Uniprot) or patient-specific exomes) to score peptides of fixed lengths using a sliding window. Substitution matrices are made by determining the frequency of all amino acids per position of the peptide. A cutoff of 0.1% frequency for an amino acid at a given position may be instituted to remove noise.

In certain aspects, the c-PPM outputs may be used to produce substitution matrices from all rounds 3 of yeast-display library selection and used to search one or more proteome databases to score peptides of fixed lengths using a sliding window. Substitution matrices are made by determining the frequency of all amino acids in the display peptides. In certain aspects, the substitution matrices are made by determining the frequency of all amino acids per position of the peptide. The scores of the peptides may calculated as the product of amino acid frequencies at each position.

A machine learning model may be used to compare the yeast-display library peptide sequences with one or more proteome databases.

Preferably, the machine learning model considers peptides as whole entities rather than taking each individual position of the peptide as independent of every other. Sequencing data including peptide sequences and round counts may be pre-processed in R to remove any peptide sequences that have fewer than a certain number of counts across all rounds. In certain aspects, the data is normalized by multiplying each round count by the average number of counts across the rounds and then divided by the number of counts in a given round. An adapted fitness score may be used to score each peptide in the library derived from a fitness function represented by an exponential curve fit to each peptide through the normalized round counts.

The model may be generated using the fitness scores for each peptide and the peptides represented as a 20×L matrix, where L is the length of the peptide sequence. The 20 rows of the matrix relate to the 20 possible amino acids. Amino acids are represented as a one-hot vector, in which a vector contains a single 1 with the remaining being 0s. The matrix representing the peptide may be flattened to a feature vector of length 20×L for use in training a neural network. The one-hot matrix may be used as input and the fitness scores used as output.

Preferably, the machine learning model models the competitive growth environment of the peptide sequences across all rounds of selection. This provides a >20-fold data augmentation when compared with prior methods. In certain aspects, module 1 employs transfer learning to train the machine learning model. Transfer learning involves training the machine learning model using one or more models that have already been trained to identify potential TCR ligands in a database that correlate to the selected yeast-display library peptide sequences. Preferably, the machine learning model is trained using one or more highly-trained long short-term memory (LSTM) and transformer embedding models. LSTM is an artificial recurrent neural network (RNN) architecture. Unlike feedforward neural networks, such as multi-layer perceptron neural networks, LSTM uses feedback connections. In feedback neural networks, connections may form cycles or loops such that learned information can move throughout layers of the network. In contrast, feedforward layers of the network do not form a cycle, and data can only move in the forward direction. This distinction means that feedforward neural networks are capable of capturing dynamic temporal information.

One potential drawback of feedback neural networks is that they can be complex and slow, as the process data in sequence. To overcome this disadvantage, transformer embedding can be used in conjunction with the LSTM model. Transformer embedding models are able to process sequential data even when it is not provided sequentially to the model. Thus, the transformer embedding allows the advantages of a feedback model to proceed efficiently.

In certain aspects, prior yeast-display library binding results are used to train a machine learning model. Assigning training data for a training data set may be random or not completely random. One or more criteria may be used during the assignment. Any suitable method may be used to assign the data to the training or testing data sets.

A training module may train the machine learning module by extracting a feature set from the training data set 410 according to one or more feature selection techniques. The training module may train the machine learning model by extracting a feature set from the training data set that includes statistically significant features of positive examples (e.g., mimotopes that bind a TCR) and statistically significant features of negative examples. The training module may extract a feature set from the training data set in a variety of ways. The training module extract features multiple times. Subsequent rounds of feature extraction may use the same or different feature extraction techniques.

The training module may use the feature set(s) to build one or more machine learning-based classification models configured to identify potential antigen candidates for a TCR of interest by comparing the yeast-display peptide library sequences with peptide sequences in a proteome database.

After the training module generates a feature set(s), the training module may generate a machine learning-based classification model based on the feature set(s).

The extracted features may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like.

The candidate feature(s) and the machine learning module may predict potential endogenous or exogenous antigens for a TCR of interest. The result for each potential TCR antigen may include a confidence level that corresponds to a likelihood or a probability that the receptor sequence will bind to a peptide. The top performing candidate feature(s) may be used to predict whether a particular antigen will bind to a TCR of interest. For example, a potential peptide ligand sequence for a TCR of interest may be determined. The new TCR sequence may be provided to the machine learning module which may, based on the top performing candidate feature, classify the new potential peptide as either binding (yes) or not binding (no) to a TCR/TCRm of interest.

In certain aspects, the training module uses supervised, unsupervised, and/or semi-supervised (e.g., reinforcement based) machine learning-based classification models. In certain aspects, training is done in Lua with the Torch package.

In certain aspects, the resulting model is used to score given peptides from the proteome database (e.g., Uniprot) patient-specific exomes using peptides isolated from an L-length sliding window converted to one-hot matrices for neural network input. P-values and Bonferroni-corrected p-values were calculated for each peptide, representing the probability of randomly selecting, from the whole proteome, a peptide with fitness score as high as or higher than the scored peptide.

The analysis of the yeast-display peptides may reveal that the selected set of peptide ligands exhibit a restricted choice of amino acids at residues, e.g., the residues that contact the TCR, which information can be input into a machine learning algorithm as described that can be used to analyze public databases for all peptides that meet the criteria for binding, and which provides a set of peptides that meet these criteria. The first module may also use the peptide sequence to identify sequence or binding motifs in the peptide sequences, which may identify superbinders-peptide sequences with a higher affinity for a TCR than endogenous antigen.

In certain aspects, one or more quality control and/or validation steps may be used. Quality control steps may, for example, provide enhanced identification of biological versus artefactual TCR antigen specificity peptide groups. The quality control may be improved using machine learning trained on the historical outcome of screens, e.g., peptide predictions validated using wet lab assays and/or further iterations using module 1.

Unlike prior methods that use exome data to identify patient-specific neoantigens that can serve as potential targets of the T cell immune response, the presently disclosed systems and methods may provide an unbiased interrogation of TCR specificities of an immune response, which relies on a physical interaction between the TCR and pHLA. This ligand identification method may be especially important in cancers that have low mutational burden, in which neoantigen targets may not be as prevalent compared to wildtype antigens.

In certain aspects, binding predictions determined may be validated or analyzed further, for example, using another yeast peptide display assay. This assay may use a display library where the peptides include sequences and/or sequence motifs, for example by using anchor peptides. Similarly, sequences and/or sequence motifs of interest may be identified in, for example, a public database.

As explained herein, the methods and systems of the invention may incorporate pHLA yeast-display libraries, where the yeast cells display a ligand that potentially binds with a TCR of interest. In certain aspects, the peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. It will be appreciated that a fully random library would represent an extraordinary number of possible combinations. In preferred methods, the diversity is limited at the residues that anchor the peptide to the MHC binding domains, which are referred to herein as MHC anchor residues. The position of the anchor residues in the peptide may be determined by the specific MHC binding domains. Class

I binding domains can have anchor residues at the P2 position, and at the last contact residue. Class II binding domains have an anchor residue at P1, and depending on the allele, at one of P4, P6 or P9. For example, the anchor residues for IE^{k} are P1 {1,L, V} and P9 {K}; the anchor residues for HLA-DR15 are P1 {1,L, V} and P4 {F, Y}. Anchor residues for DR alleles are shared at P1, with allele-specific anchor residues at P4, P6, P7, and/or P9.

### TCR and Epitope Expression

In some embodiments, the binding domains of a major histocompatibility complex protein may be soluble domains of Class II alpha and beta chain. In certain aspects, the binding domains are subjected to mutagenesis and selected for amino acid changes to enhance the solubility of the single chain polypeptide, without changing the peptide binding contacts. In certain specific embodiments, the binding domains are HLA-DR4a comprising the set of amino acid changes {M36L, V132M}; and HLA-DR4|3 comprising the set of amino acid changes {H62N, D72E}. In certain specific embodiments, the binding domains are HLA-DR15a comprising the set of amino acid changes {F12S, M23K}; and HLA-DR15|3 comprising the amino acid change {P11 S}. In certain specific embodiments, the binding domains are H2 IE^{k}a comprising the set of amino acid changes {I8T, F12S, L14T, A56V} and H2 IE^{k}|3 comprising the set of amino acid changes {W6S, L8T, L34S}.

In some embodiments, the binding domains of a major histocompatibility complex protein comprise the alpha 1 and alpha 2 domains of a Class I MHC protein, which are provided in a single chain with β2 microglobulin. In some such embodiments the Class I protein has been subjected to mutagenesis and selected for amino acid changes that enhance the solubility of the single chain polypeptide, without altering the peptide binding contacts. In certain specific embodiments, the binding domains are HLA-A2 alpha 1 and alpha 2 domains, comprising the amino acid change {Y84A}. In certain specific embodiments, the binding domains are H2-L^{d} alpha 1 and alpha 2 domains, comprising the amino acid change {M31 R}. In certain specific embodiments the binding domains are HLA-B57 alpha 1, alpha 2 and alpha 3 domains, comprising the amino acid change {Y84A}.

Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self) or as being derived from another source of hematopoietic reconstituting cells (” non-self). Two main classes of HLA antigens are recognized: HLA class I and HLA class II.

The MHC proteins used in the libraries and methods of the invention may be from any mammalian or avian species, e.g., primate sp., particularly humans; rodents, including mice, rats and hamsters; rabbits; equines, bovines, canines, felines; etc. Of particular interest are the human HLA proteins, and the murine H-2 proteins. Included in the HLA proteins are the class II subunits HLA-DPa, HLA-DPβ, HLA-DQα, HLA-DQβ, HLA-DRα and HLA-DRβ, and the class I proteins HLA-A, HLA-B, HLA-C, and β₂-microglobulin. Included in the murine H-2 subunits are the class I H-2K, H-2D, H-2L, and the class II I-Aα, I-Aβ, 1-Ea and I-Eβ, and β₂-microglobulin.

The MHC binding domains are typically a soluble form of the normally membrane-bound protein. The soluble form is derived from the native form by deletion of the transmembrane domain. Conveniently, the protein may be truncated, removing both the cytoplasmic and transmembrane domains. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ, and DR) exists in different alleles. Current nomenclature for HLA alleles is designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1 171-6, herein specifically incorporated by reference.

The numbering of amino acid residues on the various MHC proteins and variants may be made to be consistent with the full-length polypeptide. Boundaries may be offset to either be the end of the MHC peptide binding domain (as judged by examining crystal structures) for the ‘mini’ MHCs, and the end of the Beta2/Alpha2/Alpha3 domains as judged by structure and/or sequence for the ‘full length’ MHCs.

The function of MHC molecules is to bind peptide fragments derived from pathogens and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Class II binding domains generally comprise the a1 and a2 domains for the a chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind peptide ligands.

In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

In certain specific embodiments, the binding domains are an HLA-DR allele. The HLA-DRA binding domains can be combined with any one of the HLA-DRB binding domains. In certain such embodiments, the HLA-DRA allele is paired with the binding domains of an HLA-DRB4 allele. The HLA-DRB4 allele can be selected from the publicly available DRB4 alleles. In other such embodiments the HLA-DRA allele is paired with the binding domains of an HLA-DRB15 allele. The HLA-DRB15 allele can be selected from the publicly available DRB15 alleles. In other embodiments the Class II binding domains are an H2 protein, e.g., I-Aα, I-Aβ, 1-Ea and I-Eβ. In some such embodiments, the binding domains are H2 IE^{k}a which may comprise the set of amino acid changes {I8T, F12S, L14T, A56V}; and H2 IE^{k}|3 which may comprise the set of amino acid changes {W6S, L8T, L34S}.

Class I HLA/MHC. For class I proteins, the binding domains may include the a1, a2 and a3 domain of a Class I allele, including without limitation HLA-A, HLA-B, HLA-C, H-2K, H-2D, H-2L, which are combined with β₂-microglobulin. Not more than about 10, usually not more than about 5, preferably none of the amino acids of the transmembrane domain will be included. The deletion will be such that it does not interfere with the ability of the domains to bind peptide ligands.

In certain specific embodiments, the binding domains are HLA-A2 binding domains, e.g., comprising at least the alpha 1 and alpha 2 domains of an A2 protein. A large number of alleles have been identified in HLA-A2, including without limitation HLA-A*02:01:01:01 to HLA-A*02:478, which sequences are available at, for example, Robinson et al. (201 1), The IMGT/HLA database. Nucleic Acids Research 39 Suppl 1:D1 171-6. Among the HLA-A2 allelic variants, HLA-A*02:01 is the most prevalent. The binding domains may comprise the amino acid change {Y84A}.

In certain specific embodiments, the binding domains are HLA-B57 binding domains, e.g., comprising at least the alpha 1 and alpha 2 domains of a B57 protein. The HLA-B57 allele can be selected from the publicly available B57 alleles.

T cell receptor refers to the antigen/MHC binding heterodimeric protein product of a vertebrate, e.g., mammalian, TCR gene complex, including the human TCR α, β, γ and δ chains. For example, the complete sequence of the human β TCR locus has been sequenced, as published by Rowen et al. (1996) Science 272(5269):1755-1762; the human a TCR locus has been sequenced and resequenced, for example see Mackelprang et al. (2006) Hum Genet. 1 19(3):255-66; see a general analysis of the T-cell receptor variable gene segment families in Arden Immunogenetics, 1995; 42(6):455-500; each of which is herein specifically incorporated by reference for the sequence information provided and referenced in the publication.

The multimerized T cell receptor for selection in module 1 may be a soluble protein comprising the binding domains of a TCR of interest, e.g., TCRα/β, TCRy/δ. The soluble protein may be a single chain, or more usually a heterodimer. In some embodiments, the soluble TCR is modified by the addition of a biotin acceptor peptide sequence at the C terminus of one polypeptide. After biotinylation at the acceptor peptide, the TCR can be multimerized by binding to biotin binding partner, e.g., avidin, streptavidin, traptavidin, neutravidin, etc. The biotin binding partner can comprise a detectable label, e.g., a fluorophore, mass label, etc., or can be bound to a particle, e.g., a paramagnetic particle. Selection of ligands bound to the TCR can be performed by flow cytometry, magnetic selection, and the like as known in the art.

Peptide ligands of the TCR are peptide antigens against which an immune response involving T lymphocyte antigen specific response can be generated. Such antigens include antigens associated with autoimmune disease, infection, foodstuffs such as gluten, etc., allergy or tissue transplant rejection. Antigens also include various microbial antigens, e.g., as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A: MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, a-fetoprotein; etc.; and self-antigens, e.g. her-2/neu; carcinoembryonic antigen, muc-1, and the like.

Conventional methods of assembling the coding sequences can be used in the methods and systems of the invention. In order to generate the diversity of peptide ligands, randomization, error prone PCR, mutagenic primers, and the like as known in the art are used to create a set of polynucleotides. The library of polynucleotides is typically ligated to a vector suitable for the host cell of interest. In various embodiments the library is provided as a purified polynucleotide composition encoding the P-L₁-β-L₂-a-L₃-T polypeptides; as a purified polynucleotide composition encoding the P-L β-L₂-a-L₃-T polypeptides operably linked to an expression vector, where the vector can be, without limitation, suitable for expression in yeast cells; as a population of cells comprising the library of polynucleotides encoding the P-L β-L₂-a-L₃-T polypeptides, where the population of cells can be, without limitation yeast cells, and where the yeast cells may be induced to express the polypeptide library.

The term “specificity” refers to the proportion of negative test results that are true negative test result. Negative test results include false positives and true negative test results.

The term “sensitivity” is meant to refer to the ability of an analytical method to detect small amounts of analyte. Thus, as used here, a more sensitive method for the detection of amplified DNA, for example, would be better able to detect small amounts of such DNA than would a less sensitive method. “Sensitivity” refers to the proportion of expected results that have a positive test result.

The term “reproducibility” as used herein refers to the general ability of an analytical procedure to give the same result when carried out repeatedly on aliquots of the same sample.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos Biosciences Corporation (Cambridge, MA) as described in U.S. application Ser. No. 11/167,046, and U.S. Pat. Nos. 7,501,245; 7,491,498; 7,276,720; and in U.S. Patent Application Publication Nos. US20090061439; US20080087826;US20060286566; US20060024711; US20060024678; US20080213770; and US20080103058; 3) Applied Biosystems (e.g. SOLID sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Expression construct: Sequences encoding a peptide disclosed herein or a TCR disclosed herein may be introduced on an expression vector, e.g., into a cell to be engineered, as a vaccine, etc. The TCR sequence may be introduced at the site of the endogenous gene, e.g., using CRISPR technology (see, for example Eyquem et al. (2017) Nature 543:1 13-1 17; Ren et al. (2017) Protein & Cell 1-10).

Amino acid sequence variants are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

The nucleic acid encoding the sequence is inserted into a vector for expression and/or integration. Many such vectors are available. For example, the CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

The sequences may be produced recombinantly as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors may contain a selection gene, also termed a selectable marker.

This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, a-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryotic, yeast, or other eukaryotic cells described above. Examples of useful mammalian host cell lines are mouse L cells (L-M[TK-], ATCC#CRL-2648), monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10): Chinese hamster ovary cells/-DHFR (CHO); mouse Sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL. 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, etc. can be transfected with the above-described expression vectors. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition can be a vector suitable for introducing the sequence into a targeted cell for expression. The label on or associated with the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

Also provided herein in certain aspects are libraries of polypeptides comprising or consisting essentially of at least one of the SCT polypeptides of the present disclosure and/or at least one of the polypeptide compositions of the present disclosure. In certain aspects, the libraries are peptide-HLA-B*35 libraries. It will be appreciated that a fully random library would represent an extraordinary number of possible combinations. In preferred methods, the target peptides (i.e., peptide ligands) of the library are diversified (e.g., randomized or not randomized) at multiple positions, and the diversity is limited at the residues that anchor the peptide to the MHC binding domains, which are referred to herein as MHC anchor residues. The position of the anchor residues in the peptide are determined by the specific MHC binding domains. HLA-B*35 binding domains have anchor residues at the P2 position, and at the last contact residue (e.g., the P9 position). In certain aspects, the target peptide (i.e., peptide ligand) of the SCT polypeptides have NNK codons at positions 1, 3-8 were used to diversity the peptide, and known anchor residues position 2 and position 9 were restricted to allowed amino acids. In certain aspects, the libraries comprise SCT polypeptides comprising HIV(Pol448-456), β2 microgrobulin, and an HLA-B*35 alpha chain. In certain aspects, the libraries comprise SCT polypeptides comprising NY-ESO-1 [e.g., NY-ESO-1(94-102)], β2 microgrobulin, and an HLA-B*35 alpha chain.

In certain aspects, the library comprises at least 10⁶, at least 10⁷, more usually at least 10⁸, or at least 10⁹ different target peptides (i.e., peptide ligands) that are displayed on cell surface in the context of the HLA-B*35 allele. In some certain, the libraries can be used to identify the recognition properties of ligands of HLA-B*35-restricted T cell receptors.

The different target peptides (i.e., peptide ligands) of the libraries may be created by any methods known in the art, including error prone mutagenesis, and a gene editing system, e.g., clustered, regularly interspaced, short, palindromic repeats (CRISPR)/CRISPR-associated (Cas) system, transcription activator-like effector nucleases (TALEN) system, zinc-finger protein (ZNF) system, or Transposase system into cells.

Further provided herein in certain embodiments are pharmaceutical compositions comprising or consisting essentially of at least one of the SCT polypeptides of the present disclosure and/or at least one of the polypeptide compositions of the present disclosure.

Also provided herein in certain embodiments are cells comprising or consisting essentially of at least one of the SCT polypeptides of the present disclosure and/or at least one of the polypeptide compositions of the present disclosure. In some embodiments, the cells are yeast cells, e.g., Saccharomyces cerevisiae cells. In other embodiments, the cells are mammalian cells or insect cells.

In some embodiments, a target peptide is displayed on a cell surface by modifying the cell with the SCT polypeptides or the SCT polypeptide compositions of the present disclosure. Such modification of the cell with the SCT polypeptides or the SCT polypeptide compositions may be performed by a number of methods well known in the art, including, but not limited to, transfection, electroporation, recombination, transformation, transduction, or CRISPR gene editing.

In some embodiments, expression of the SCT polypeptides or the SCT polypeptide compositions is induced in the cells. Inducing expression of the SCT polypeptides or the SCT polypeptide compositions may be achieved by methods well known in the art, including inducing cell proliferation, expressing the SCT polypeptides or the SCT polypeptide compositions under an inducible promoter, targeting promotor sequences, or gene editing

Further provided herein in certain embodiments are first nucleic acids comprising or consisting essentially of a second nucleic acid encoding at least one of the SCT polypeptides of the present disclosure and/or at least one of the polypeptide compositions of the present disclosure.

Also provided herein in certain embodiments are expression vectors comprising or consisting essentially of at least one of the nucleic acids of the present disclosure. In some embodiments, the nucleic acids of the present disclosure are located under an inducible promoter in the expression vector, such that the expression of the nucleic acids is inducible.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are methods comprising or consisting essentially of preparing one or more polypeptides selected from the group consisting of the SCT polypeptides of the present disclosure and the polypeptide compositions of the present disclosure, the method comprising co-expressing protein disulfide isomerase with one or more of the polypeptides of the present disclosure, culturing the cells of the present disclosure, and isolating the one or more polypeptides from the cell or a culture medium thereof.

In some embodiments, disulfide bond formation can be enhanced with co-expression of protein disulfide isomerase (PDI).

Further provided herein in certain embodiments are methods of displaying a target peptide on a cell surface, the method comprising modifying the cell with a first nucleic acid comprising or consisting essentially of a second nucleic acid encoding at least one of the SCT polypeptides and/or at least one of the polypeptide compositions of the present disclosure. Modifying the cell with the SCT polypeptides or the polypeptide compositions may be performed by a number of methods well known in the art, including, but not limited to, transfection, electroporation, recombination (e.g., homologous recombination), transformation, transduction, or gene editing (e.g., introducing a CRISPR-Cas9 system, a TALEN system, or a ZNF system into cells). An exemplary gene editing system comprises a nuclease and a guide RNA. A CRISPR system comprises a CRISPR nuclease (e.g., CRISPR (clustered regularly interspaced short palindromic repeats)-associated (Cas) endonuclease or a variant thereof, such as Cas9) and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. As used herein, a “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

In some embodiments, the methods optionally include inducing expression of the SCT polypeptides and/or the at least one of the polypeptide compositions by, for example, inducing cell proliferation, expressing the SCT polypeptides or the SCT polypeptide compositions under an inducible promoter and activating the promotor, targeting promotor sequences, or gene editing. In some embodiments, the cells are yeast cells, e.g., Saccharomyces cerevisiae cells. In some embodiments, the cells are mammalian cells or insect cells.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are in vitro methods for producing activated T cells, comprising or consisting essentially of contacting T cells with one or more of the SCT polypeptides of the present disclosure and/or one or more of the polypeptide compositions of the present disclosure.

Further provided herein in certain embodiments are activated T cells, produced by the methods of the present disclosure, that selectively recognize a cell expressing one or more peptides selected from the group consisting of the target peptides of the present disclosure.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized, for example, by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Publication Nos. US20090061439; US20080087826; US20060286566; US20060024711; US20060024678; US20080213770; and US20080103058; 3) Applied Biosystems (e.g. SOLID sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Patent Publication Nos. US20090029385; US20090068655; US20090024331; and US20080206764.

The results of any of modules 1, 2, or 3 can be used to engineer antigen-presenting cells. Antigen-presenting cells (APCs) may include cells that present complexes formed between HLA antigens and the peptides on its surface. APCs may be obtained by contacting the peptides, or the nucleotides encoding the peptides, and can be prepared from subjects who are the targets of treatment and/or prevention, and can be administered as vaccines by themselves or in combination with other drugs including the peptides, exosomes, or cytotoxic T cells. The APCs are not limited to any kind of cells and includes dendritic cells (DCs), Langerhans cells, macrophages, B cells, and activated T cells, all of which are known to present proteinaceous antigens on their cell surface so as to be recognized by lymphocytes. Since DC is a representative APC having the strongest CTL inducing action among APCs, DCs find particular use as the APCs.

Cells may be engineered to express a TCR of interest, such as a TIL, or to respond to a peptide antigen provided herein. A number of different cell types are suitable for engineering, particularly T cells or NK cells. In some embodiments the cells for engineering are autologous. In some embodiments the cells are allogeneic.

A T cell stimulated against any of the peptides disclosed herein can be used as vaccines similar to the peptides. Thus, the present invention provides isolated T cells that are stimulated by any of the present peptides. Such T cells can be obtained by (1) administering to a subject or (2) contacting (stimulating) subject-derived APCs, and CD8-positive cells, or peripheral blood mononuclear leukocytes in vitro with the peptide. T cells, which have been stimulated by stimulation from APCs that present the peptides, can be derived from subjects who are targets of treatment and/or prevention, and can be administered by themselves or in combination with other drugs including the peptides or exosomes for the purpose of regulating effects. The obtained T cells act specifically against target cells presenting the peptides, for example, the same peptides used for priming. The target cells can be cells that express endogenously, or cells that are transfected with genes, and cells that present the peptides on the cell surface due to stimulation by these peptides can also become targets of attack.

In certain aspects, the engineered cell is a T cell. The term “T cells” refers to mammalian immune effector cells that may be characterized by expression of CD3 and/or T cell antigen receptor, which cells can be engineered to express a TCR provided herein or stimulated to respond to a peptide provided herein. In some embodiments the T cells are selected from naive CD8⁺ T cells, cytotoxic CD8⁺ T cells, naive CD4⁺ T cells, helper T cells, c.g., T_{H}1, T_{H}2, T_{H}9, T_{H}1 1, T_{H}22, T_{FH}; regulatory T cells, e.g., T_{R}1, natural T_{Reg}, inducible T_{Reg}; memory T cells, e.g., central memory T cells, T stem cell memory cells (T_{S}CM)-effector memory T cells, NKT cells, γδT cells. In some embodiments, the engineered cells comprise a complex mixture of immune cells, e.g., tumor infiltrating lymphocytes (TILs) isolated from an individual in need of treatment. In certain aspects, T cells are contacted with a peptide in vitro, i.e., where the T cells are then transferred to a recipient.

Effector cells may include autologous or allogeneic immune cells having cytolytic activity against a target cell, including without limitation tumor cells. Effector cells may be obtained by engineering peripheral blood lymphocytes (PBL) in vitro, then culturing with a cytokine and/or antigen combination that increases activation. The cells may be optionally separated from non-desired cells prior to culture, prior to administration, or both. Cell-mediated cytolysis of target cells by immunological effector cells is believed to be mediated by the local directed exocytosis of cytoplasmic granules that penetrate the cell membrane of the bound target cell.

Cytotoxic T lymphocytes (CTL) reactive to tumor cells are specific effector cells for adoptive immunotherapy and are of interest for engineering by priming with peptides disclosed herein, or engineering to express a TCR disclosed herein. Induction and expansion of CTL is antigen-specific and MHC restricted.

T cells collected from a subject may be separated from a mixture of cells by techniques that enrich for desired cells, or may be engineered and cultured without separation. An appropriate solution may be used for dispersion or suspension. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected cell population may be used or may be frozen at liquid nitrogen temperatures and stored, being thawed and capable of being reused. The cells will usually be stored in 10% DMSO, 50% FCS, 40% RPMI 1640 medium.

### Machine Learning Algorithms

The present invention benefits from the ability to predict epitopes of TCRs using by a machine-learning (ML) algorithm or statistical algorithm. This includes not only systems and methods using ML, but also the training of ML systems to increase their accuracy and predictive value.

Machine learning is branch of computer science in which machine-based approaches are used to make predictions. (Bera et al., Nat Rev Clin Oncol., 16(11):703-715 (2019)). ML-based approaches involve a system learning from data fed into it, and using this data to make and/or refine predictions. Id. Machine learning is distinct from traditional, rule-based or statistics-based program models. (Rajkomar et al., N Engl J Med, 380:1347-58 (2019)). Rule-based program models require software engineers to code explicit rules, relationships, and correlations. Id. For example, in the medical context, a physician may input a patient's symptoms and current medications into a rule-based program. In response, the program will provide a suggested treatment based upon preconfigured rules.

In contrast, and as a generalization, in ML a model learns from examples fed into it. Id. Over time, the ML model learns from these examples and creates new models and routines based on acquired information. Id. As a result, an ML model may create new correlations, relationships, routines or processes never contemplated by a human. A subset of ML is deep learning (DL). (Bera et al. (2019)). DL uses artificial neural networks. A DL network generally comprises layers of artificial neural networks. Id. These layers may include an input layer, an output layer, and multiple hidden layers. Id. DL has been shown to learn and form relationships that exceed the capabilities of humans. (Rajkomar et al. (2019)).

By combining the ability of ML, including DL, to develop novel routines, correlations, relationships and processes for epitope prediction, the methods and systems of the disclosure can provide an exhaustive epitope profile for a TCR of interest, for example epitopes that include wildtype human sequences, patient-specific neoantigens, shared neoantigens, spliced peptides, human endogenous retroviruses (HERVs), long interspersed nuclear elements (LINEs), frameshifts, gene fusions, alternative splicing, aberrant translations, alternative promoters, human-viral targets, and human-bacterial targets.

Any suitable machine learning system may be used. For example, the machine learning systems may learn in a supervised manner, an unsupervised manner, a semi-supervised manner, or through reinforcement learning.

In supervised learning models, the machine learning system is given training data categorized as input variables paired with output variables from which to learn patterns and make inferences in order to generate a prediction on previously unseen test data. Supervised models replicate an identified mapping system and recognize and respond to patterns in data without explicit instructions Supervised models are advantageous for performing classification tasks, in which data inputs are separated into categories. Supervised models are also advantageous for regression tasks, in which the output variable is a real value, such as a price or a volume. The accuracy of a supervised model is easy to evaluate, because there is a known output variable to which the model is optimizing.

In an unsupervised model or autonomous model, the machine learning system is only given input training data without paired output data from which to identify patterns autonomously. Unsupervised models identify underlying patterns or structures in training data to make predictions for test data. Unsupervised models are advantageous for clustering data, anomaly detection, and for independently discovering rules for data. The accuracy of unsupervised models is harder to evaluate because there is no predefined output variable to which the system is optimizing. Autonomous models may employ periods of both supervised and unsupervised learning in order to optimize predictions.

In semi-supervised models, the machine learning system is given training data comprising input variables, with output variable pairs available for only a limited pool of the input variables. The model uses the input variables with known output variables and the remaining input training data to learn patterns and make inferences in order to generate a prediction on previously unseen test data. A semi-supervised model may query the user for additional paired output data based on unlabeled data.

In a reinforcement learning model, the machine learning system is given neither input variables nor output variables. Rather, the model provides a “reward” condition and then seeks to maximize the cumulative reward condition by trial and error. A common reinforcement learning model is a Markov Decision Process.

A common supervised learning model is a “decision tree.” Decision trees are non-parametric supervised learning models that use simple decision rules to infer a classification for test data from the features in the test data. In classification trees, test data take a finite set of values, or classes, whereas in regression trees, the test data can take continuous values, such as real numbers. Decision trees have some advantages in that they are simple to understand and can be visualized as a tree starting at the root (usually a single node) and repeatedly branch to the leaves (multiple nodes) that are associated with the classification. See Criminisi, 2012, Decision Forests: A unified framework for classification, regression, density estimation, manifold learning and semi-supervised learning, Foundations and Trends in Computer Graphics and Vision 7(2-3):81-227, incorporated by reference.

Another supervised learning model is a “support-vector machine” (SVM) or “support-vector network.” SVMs are supervised learning models for classification and regression problems. When used for classification of new data into one of two categories, for example a predicted epitope of a TCR or a predicted non-epitope of a TCR, an SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines, Numerical Recipes: The Art of Scientific Computing (3rd ed.). New York: Cambridge University (2007), incorporated herein by reference. Where output variables are unavailable for input variables in the training data, SVMs can be designed as unsupervised learning models using support vector clustering. See Ben-Hur, 2001, Support Vector Clustering, J Mach Learning Res 2:125-137, incorporated by reference.

Some models rely on clustering training data and test data to find patterns and make predictions. A “k-nearest neighbor” (k-NN) model is a non-parametric supervised learning model for classification and regression problems. A k-nearest neighbor model assumes that similar data exists in close proximity, and assigns a category or value to each data point based on the k nearest data points. k-NN models may be advantageous when the data has few outliers and can be defined by homogeneous features. A common unsupervised learning model that uses clustering is a “k-means” clustering model. A k-means model looks to find clusters of data in input data and test data. K-means models are advantageous when a defined number of clusters are known to exist in the data and are also advantageous when the test data has few outliers and can be defined homogeneous features. Additional models that cluster training data include, for example, farthest-neighbor, centroid, sum-of-squares, fuzzy k-means, and Jarvis-Patrick clustering.

Bayesian algorithms can also be used to find patterns in training and test data to make predictions. Bayesian networks are probabilistic graphical models that represent a set of random variables and their conditional dependencies via directed acyclic graphs (DAGs). The DAGs have nodes that represent random variables that may be observable quantities, latent variables, node unknown parameters or hypotheses. Edges represent conditional dependencies; nodes that are not connected represent variables that are conditionally independent of each other. Each is associated with a probability function that takes, as input, a particular set of values for the node's parent variables, and gives (as output) the probability (or probability distribution, if applicable) of the variable represented by the node.

Regression analysis is another statistical process that can be used to find patterns in training and test data to make predictions. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Trained machine learning models can become “stable learners.” A stable learner is a model that is less sensitive to perturbation of predictions based on new training data. Stable learners can be advantageous where test data is stable, but can be less advantageous where the system needs to continually improve performance to accurately predict new test data.

Several machine learning system types can be combined into final predictive models known as ensembles. Ensembles can be divided into two types, homogenous ensembles and heterogeneous ensembles. Homogenous ensembles combine multiple machine learning models of the same type. Heterogeneous ensembles combine multiple machine learning models of different types. Ensembles can provide the advantage of being more accurate than any of the individual member models (“members”) in the ensemble. The number of members combined in an ensemble may impact the accuracy of a final prediction. Accordingly, it is advantageous to determine the optimal number of members when designing an ensemble system.

Ensembles may combine or aggregate outputs from individual members using “voting”-type methods for classification systems and “averaging”-type methods for regression systems.

In a “majority voting” method, each member makes a prediction for test data and the prediction that receives more than half of the votes is the final output for the ensemble. If none of the predictions receives more than half of the votes, it may be determined that the ensemble is unable to make a stable prediction. In a “plurality voting” method the most voted prediction, even if receiving less than half of the votes, may be considered the final output for the ensemble. In a “weighted voting” method, the votes of more accurate members are multiplied by a weight afforded each member based on its accuracy.

In a “simple averaging” method, each member makes a prediction for test data and the average of the outputs is calculated. This method reduces overfit and can be advantageous in creating smoother regression models. In a “weight averaging” method, the prediction output of each member is multiplied by a weight afforded each member based on its accuracy. Voting methods, averaging methods, and weighted methods can be combined to improve the accuracy of ensembles.

Members within an ensemble can each be trained independently or new members can be trained utilizing information from previously trained members. In a “parallel ensemble”, the ensemble seeks to provide greater accuracy than individual members by exploiting the independence between members, for example, by training multiple members simultaneously and aggregating the outputs from members. In “sequential ensemble systems”, the ensemble seeks to provide greater accuracy than individual members by exploiting the dependence between members, for example, by utilizing information from a first member to improve the training of a second member and weighting outputs from members.

Overall accuracy for ensembles can also be optimized by using ensemble meta-algorithms, for example a “bagging” algorithm to reduce variance, a “boosting” algorithm to reduce bias, or a “stacking” algorithm to improve predictions.

Boosting algorithms reduce bias and can be used to improve less accurate, or “weak learning” models. A member may be considered a “weak learning” model if it has a substantial error rate, but its performance is non-random, for example an error rate of 0.5 for binary classifications. Boosting algorithms incrementally build the ensemble by training each member sequentially with the same training data set, examining prediction errors for test data, and assigning weights to training data based on the difficulty for members to make an accurate prediction. In each sequential member trained, the algorithm emphasizes training data that previous members found difficult. Members are then weighted based on the accuracy of their prediction outputs in view of the weight applied to their training data. The predictions from each member may be combined by weighted voting-type or weighted averaging-type methods. Boosting algorithms are advantageous when combining multiple weak learning models. Boosting algorithms may, however, result in over-fitting test data to training data.

Examples of boosting algorithms include AdaBoost, gradient boosting, eXtreme Gradient Boost (XGBoost). See Freund, 1997, A decision-theoretic generalization of on-line learning and an application to boosting, J Comp Sys Sci 55:119; and Chen, 2016, XGBoost: A Scalable Tree Boosting System, arXiv:1603.02754, both incorporated by reference.

Bagging algorithms or “bootstrap aggregation” algorithms reduce variance by averaging together multiple estimates from members. Bagging algorithms provide each member with a random sub-sample of a full training data set, with each random sub-sample known as a “bootstrap” sample. In the bootstrap samples, some data from the training data set may appear more than once and some data from the training data set may not be present. Because sub-samples can be generated independently from one another, training can be done in parallel. The predictions for test data from each member are then aggregated, such as by voting-type or averaging-type methods.

An example of a bagging algorithm that may be utilized is a “random forest”. In a random forest the ensemble combines multiple randomized decision tree models. Each decision tree model is trained from a bootstrap sample from a training set. The training set itself may be a random subset of features from an even larger training set. By providing a random subset of the larger training set at each split in the learning process, spurious correlations that can results from the presence of individual features that are strong predictors for the response variable are reduced. By averaging predictions for test data, variance of the ensemble decreases resulting in an improved prediction. Random forests may be autonomous models and may include periods of both supervised and unsupervised learning. Bagging may be less advantageous in optimizing an ensemble combining stable learning systems, since stable learning systems tend provide generalized outputs with less variability over the bootstrap samples. See Breiman, 2001, Random Forests, Machine Learning 45:5-32, incorporated by reference.

Stacking algorithms or “stacked generalization” algorithms improve predictions by using a meta-machine learning model to combine and build the ensemble. In stacking algorithms, base member models are trained with a training dataset and generate as an output a new dataset. This new dataset is then used as a training dataset for the meta-machine learning model to build the ensemble. Stacking algorithms are generally advantageous when building heterogeneous ensembles.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Within the network, nodes are connected in layers, and signals travel from the input layer to the output layer. Each node in the input layer may correspond to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs. See WO 2016/182551, U.S. Pub. 2016/0174902, U.S. Pat. 8,639,043, and U.S. Pub. 2017/0053398, each incorporated by reference.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, cach feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

For example, a convolutional neural network (CNN) is a class of deep neural network generally designed for two-dimensional image inputs in which a signal travels from the input layer through hidden layers comprising “convolutional layers” and “fully connected layers” to the output layer. In the input layer, each pixel from a signal is mapped to a node. The input layer is connected to a convolutional layer. In a convolutional layer, each node is “sparsely connected”, that is connected to only a sub-matrix of nodes from the previous layer. The connection between the submatrix of nodes and the convolutional layer is subject to a bias term as a set of weights designed detect a given feature in the input. The submatrix and weights together are known as a “filter,” “kernel,” or “feature detector”. For a given convolutional layer, each filter is the same size and shape and applies the same set of weights. Each node in the convolutional layer is provided a summary of the weighted information from the filter as a scalar dot product. The filters are staggered from one another and may overlap such that each node in convolution layer provides a weighted summary for a different sub-matrix from the previous layer. A threshold function may be applied to each node in the convolution layer to determine whether the node will propagate the information from the filter, a function known as “squashing.”

Sliding the filter systematically across the entire input allows the filter to discover a given feature anywhere in the input. The function of sliding the filter over entire image can be controlled by the number of nodes over which the filter movies, known as the “stride” of the convolutional layer. The stride determines the distance that each filter is staggered from adjacent filters and the degree of overlap between filters. The final two-dimensional array of dot products of the convolutional layer is known as the “convolved feature,” “activation map,” or “feature map.”

In some instances, it may also be convenient to “pad” an input to a convolutional layer with zero values around the border of the input, a process known as zero-padding. Zero-padding allows the size of feature maps to be controlled. This can allow for the feature map to remain the same size as the input through multiple layers of the CNN. The function of adding zero-padding is known as “wide-convolution” versus “narrow convolution” when no zero-padding is added.

The use of multiple convolutional layers in the network allows for hierarchical decomposition of the input. Convolutional filters that operate directly on input values may learn to extract low level features, such as lines. Convolutional filters that operate on the output from earlier convolution layers may learn to extract features that are combinations of lower-level features.

A CNN may also comprise nonlinear layers (ReLU) and/or pooling or sub sampling layers. A ReLU layer receives a feature map and replaces any negative values in the feature map with a zero. The purpose of the ReLU layer is to introduce non-linearity into the CNN and is advantageous when the input data that the CNN is expected to learn and identify is non-linear. The non-linear output map from a ReLU is known as a “rectified” feature map. A pooling layer reduces the size of the feature map or rectified feature map through dimensionality reduction in a process known as “spatial pooling,” “subsampling,” or downsampling.” For example, each node in a pooling layer may be connected to a sub-matrix of nodes from a convolution or ReLU layer. Each node in the pooling layer may then provide, for example, only the highest value, average of, or sum of the values in each submatrix. Pooling layers can be advantageous to make input representations smaller and more manageable, reduce the number of parameters and computations in the network, reduce the impact of distortions in the input image, and help scale representation of the image. This may reduce training time and control overfitting in the CNN.

The final output from the convolutional, ReLU, and/or pooling layers, is provided to a fully connected layer. The fully connected layers operate under the same principles as a traditional neural network. In a fully connected layer, each node in the layer is connected to all of the nodes in a previous layer and all of the nodes in a succeeding layer. The purpose of a fully connected layer is to classify the features extracted by the convolutional layers, for example using single vector machines (SVM).

Backpropagation in CCNs involves adjusting the weights of filters based on the error rate of the CNN, known as “loss.” During backpropagation, the CNN determines the estimated loss at every node in each convolutional layer and adjusts filter weights accordingly to minimize loss. A CNN may be trained by multiple rounds of backpropagation.

A deconvolutional neural network (DNN) is another class of deep neural network designed to generate an image from a feature map or from the output from a CNN. A DNN learns and makes predictions as to the pooling, ReLU, and convolution layers that a feature map may have undergone and performs the opposite function, e.g., unpooling and deconvolution.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

The features detected by the machine learning system may be any quantity, structure, pattern, or other element that can be measured from the training data. Features may be unrecognizable to the human eye. Features may be created autonomously by the machine learning system. Alternatively, features may be created with user input.

For example, the method of predicting epitopes of a TCR may include identifying clusters of mapped randomized peptide antigen sequences and performing a probability position matrix on the sequences of each cluster. The method may also include identifying mapped randomized peptide antigen sequences that have a peptide sequence similar or identical to a peptide sequence of the target peptide.

In certain aspects, the method includes embedding the peptide activation predictions onto the latent space and identifying areas of the latent space proximal to peptide activation predictions for known TCR activators. The method may further include identifying a plurality of embedded peptide sequences in an area of the latent space proximal to the peptide activation prediction for one or more known TCR activators. In certain aspects, the step of conducting one or more analyses using the peptide binding predictions and the peptide activation predictions to obtain a cross-reactivity profile includes identifying embedded peptide sequences of the randomized peptide antigens in an area of the latent space proximal to the peptide activation predictions for known TCR activators.

### Example 1: Novel TCR Reactive Epitopes of Cancer

The presently disclosed systems and methods are able to identify peptides that stimulate naturally occurring TCRs in patients with antigen presenting disorders. The identified peptides may be more potent and/or provide lower cross-reactivity than previously identified peptides. The neoantigen epitopes have utility as DNA, RNA or peptide vaccines to stimulate particular antigen-specific T cells and generate a more immunogenic response.

The methods of the invention resulted in the discovery of novel epitopes present in subject afflicted with cancer. Specifically, tumor-infiltrating lymphocytes (TILs) from patient tumor samples were processed and sequenced. T cell receptors were screened to identify the landscape of targets that can be recognized by the TCR. Synthetic peptides were identified from the screen and machine learning algorithms used to predict and identify TCR specificities and off-target cross-reactivities. TCR specificity to identified antigens was then validated using antigen processing, T cell activation assays, and tumor killing assays to validated targets.

The combination of the yeast display platform and active machine learning systems allowed for the identification of novel cancer targets and TCRs. This included the most prevalent and immunogenic targets in solid tumors. In addition, the methods could be applied across all tumor types, including ovarian, lung, colorectal, melanoma, breast, and renal cell cancer. The active machine learning systems predict immunogenic targets with 95% accuracy.

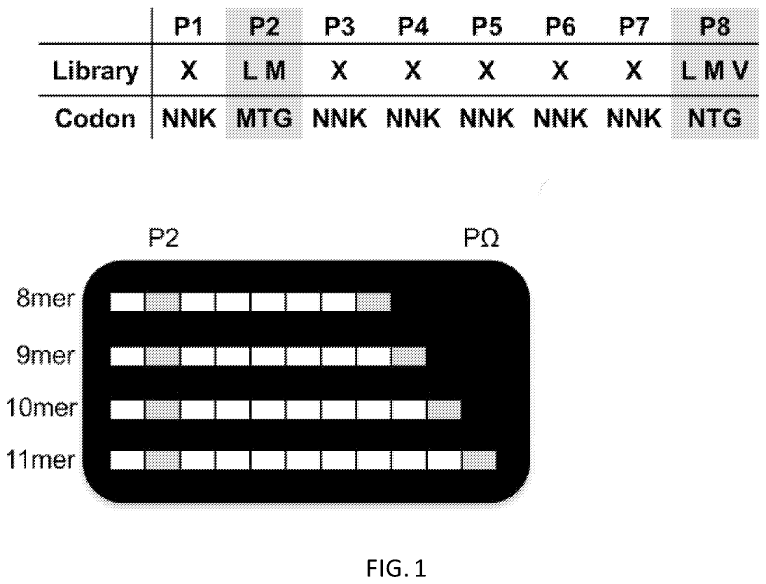

FIG. 1 depicts a method of creating a library of predicted epitopes. For example, in each of the 8-12 mer peptides, the second amino acid of the peptide (P2) was varied along with last peptide of the amino acid sequence.

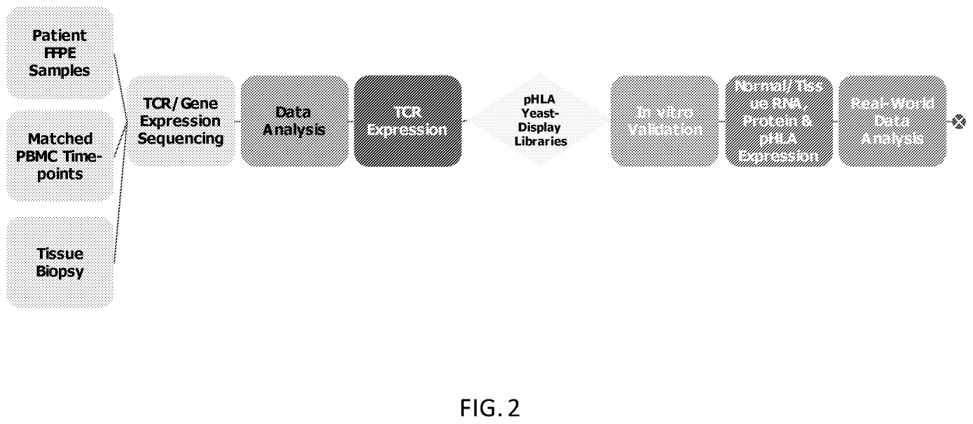

FIG. 2 depicts a flow chart of methods of the invention. Patient samples, including formalin-fixed paraffin-embedded samples, PBMCs (including matched time-points) and cells from tumor biopsies were obtained and HLAs were sequenced to generally a library of TCR. TCR were selected and genetic expression analysis conducted. Vectors were synthesized and recombination protein expression and purification conducted to produce express selected TCRs. A yeast display was then used to generate a library of peptides based on predicted targets of the TCRs. TCRs were then validated against peptide targets in vitro, through TCR activation, antigen processing, and T cell killing assays. Mass spectrometry and target expression panels were further used to validated RNA expression and IHC protein expression.

Altogether, greater than 100 patient samples, including solid tumor indications and peripheral blood checkpoint responders and non/responders, over 200,000 TCR sequences were narrowed down to 1000 screened TCRs after analyzing tumor reactivity, clonal expansion, and tumor-reactive biomarkers. From the 1000 screened TCRs, over 100 novel target epitopes were identified to be analyzed by T cell activation and killing assays, antigen processing, target biology, RNA/protein expression, tumor specificity, and tumor indications.

### Discussion of Example 1

The methods of the invention allowed for the identification of novel targets of T cell receptors that are responding to therapy using an immune-response guided approach.

The identification of novel oncology targets can be broadly applied, for example to other antigen presenting disorders. The approach involved profiling patient samples (responders and non-responders) through molecular and cellular approaches. TCRs are then prioritized and a reference proteome developed. Targets are then validated and nominated as shown in Example 1.

Unknown target antigens in oncology are the result of aberrant genetic and proteomatic alterations that are uncommon in healthy individual. The methods of the invention allow for the cataloguing and development of methods that identify an unknown target antigen, affinity of T cells to the target antigen, and to develop novel T cell receptor targets, for example, in the case of cancer, from checkpoint responder individual.

Methods of the invention allow for the analysis of new targets that comprise HERV and LINE sequences or reactivated endogenous elements. The approach allows for the addition and analysis of cis- and trans-spliced peptides and viral proteins.

Each additional analysis allows for the identification of increasingly novel targets of T cell receptors using an immune-response guided approach.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

We claim:

1. A method of identifying an epitope of an immune cell, the method comprising:
identifying T-cell receptors (TCRs) of immune cells from sequencing data obtained from a subject;
engineering a soluble TCR or T-cell expressing a TCR identified from the sequencing data;
screening the soluble TCR or engineered T-cell against a peptide library expressing predicted epitopes of the TCR of the engineered T-cell;
validating an epitope from the peptide library as an epitope of the T-cell.

2. The method of claim 1, wherein the immune cells are obtained from a subject with cancer or an auto-immune disorder.

3. The method of claim 2, wherein the immune cells from the subject are tumor or tissue-infiltrating lymphocytes or peripheral blood mononuclear cells (PBMCs).

4. The method of claim 3, wherein the immune cells are obtained from the tumor of a subject previously administered a cancer therapy.

5. The method of claim 4, wherein the cancer therapy comprises an immune checkpoint inhibitor, neoadjuvant therapy, and/or chemotherapy.

6. The method of claim 5, wherein the immune cells are obtained from the subject prior to administration of the cancer therapy and after administration of the cancer therapy.

7. The method of claim 1, wherein the peptide library is a yeast display library.

8. The method of claim 1, wherein the predicted epitopes of the TCR are predicted by a machine-learning algorithm or statistical algorithm.

9. The method of claim 8, wherein the peptide library comprises predicted epitopes selected from one or more of: wildtype human sequences, patient-specific neoantigens, shared neoantigens, spliced peptides, human endogenous retroviruses (hERVs), long interspersed nuclear elements (LINEs), aeTSAs (aberrantly expressed, tumor specific antigens), frameshifts, gene fusions, alternative splicing, aberrant translations, alternative promoters, human-viral targets, and human-bacterial targets.

10. The method of claim 9, wherein the epitopes resulting from aberrant protein splicing are cis-spliced or trans-spliced peptides.

11. The method of claim 2, wherein the immune cells are obtained from a tumor associated with one or more cancers selected from the group comprising breast cancer, cervical cancer, colorectal cancer, endometrial cancer, glioma, head and neck cancer, liver cancer, lung cancer, lymphoma, melanoma, ovarian cancer, pancreatic cancer, ovarian cancer, pancreatic cancer, prostate cancer, renal cancer, skin cancer, stomach cancer, testis cancer, thyroid cancer, and urothelial cancer.

12. The method of claim 1, wherein the peptide library comprises 8-11 mer peptides.

13. The method of claim 1, wherein the immune cells are from formalin fixed paraffin-embedded tissue.

14. The method of claim 1, wherein the validating step comprises analyzing T-cell activation, T-cell killing, mass spectrometry, functional antigen procession, and/or target expression.

15. The method of claim 14, wherein the validating step comprises analyzing T-cell killing of cells expressing the peptide by an engineered T-cell comprising the TCR.

16. A method of treating a subject afflicted with cancer or an auto-immune disorder, the method comprising providing to the subject a composition comprising an engineered T-cell or soluble TCR targeting a first epitope, wherein the first epitope was identified by the steps of:
identifying T-cell receptors (TCRs) of immune cells from sequencing data obtained from a subject;
engineering a soluble TCR or T-cell expressing a TCR identified from the sequencing data;
screening the soluble TCR or engineered T-cell against a peptide library expressing predicted epitopes of the TCR of the engineered T-cell including the first epitope; and
validating the first epitope from the peptide library as an epitope of the T-cell.

17. The method of claim 16, wherein the immune cells are obtained from a subject with cancer or an auto-immune disorder.

18. The method of claim 17, wherein the immune cells from the subject are tumor or tissue-infiltrating lymphocytes or peripheral blood mononuclear cells (PBMCs).

19. The method of claim 18, wherein the immune cells are obtained from the tumor of a subject previously administered a cancer therapy.

20. The method of claim 19, wherein the cancer therapy comprises an immune checkpoint inhibitor, neoadjuvant therapy, and/or chemotherapy.

21. The method of claim 20, where the immune cells are obtained from the subject prior to administration of the cancer therapy and after administration of the cancer therapy.

22. The method of claim 16, wherein the peptide library is a yeast display library.

23. The method of claim 16, wherein the predicted epitopes of the TCR are predicted by a machine-learning algorithm or statistical algorithm.

24. The method of claim 23, wherein the peptide library comprises predicted epitopes selected from one or more of: wildtype human sequences, patient-specific neoantigens, shared neoantigens, spliced peptides, human endogenous retroviruses (hERVs), long interspersed nuclear elements (LINEs), aeTSAs (aberrantly expressed, tumor specific antigens), frameshifts, gene fusions, alternative splicing, aberrant translations, alternative promoters, human-viral targets, and human-bacterial targets.

25. The method of claim 24 wherein the epitopes resulting from aberrant protein splicing are cis-spliced or trans-spliced peptides.

26. The method of claim 17, wherein the immune cells are obtained from a tumor associated with one or more cancers selected from the group comprising breast cancer, cervical cancer, colorectal cancer, endometrial cancer, glioma, head and neck cancer, liver cancer, lung cancer, lymphoma, melanoma, ovarian cancer, pancreatic cancer, ovarian cancer, pancreatic cancer, prostate cancer, renal cancer, skin cancer, stomach cancer, testis cancer, thyroid cancer, and urothelial cancer.

27. The method of claim 16, wherein the peptide library comprises 8-11 mer peptides.

28. The method of claim 16, wherein the immune cells are from formalin fixed paraffin-embedded tissue.

29. The method of claim 16, wherein the validating step comprises analyzing T-cell activation, T-cell killing, mass spectrometry, functional antigen procession, and/or target expression.

30. The method of claim 29, wherein the validating step comprises analyzing T-cell killing of cells expressing the peptide by an engineered T-cell comprising the TCR.
